(19) United States
(12) Patent Application Publication
(10) Pub. No.: US 2025/0180572 A1
(43) Pub. Date: Jun. 5, 2025

(54) PARTICLE, TEST PARTICLE, REAGENT, AND TEST KIT, AND DETECTION METHOD

(71) Applicant: CANON KABUSHIKI KAISHA, Tokyo (JP)

(72) Inventors: MASAKI TSUNODA, Tokyo (JP); SAKAE SUDA, Kanagawa (JP); IBUKI ASANO, Kanagawa (JP); RYO NATORI, Tokyo (JP); MASASHI KAWAMURA, Kanagawa (JP)

(21) Appl. No.: 18/956,136

(22) Filed: Nov. 22, 2024

(30) Foreign Application Priority Data
Nov. 30, 2023 (JP) 2023-203386

(51) Int. Cl. G01N 33/68; G01N 33/531

(52) U.S. Cl. CPC G01N 33/68; G01N 33/531; G01N 2333/4737

(57) ABSTRACT

A particle includes a copolymer having a structural unit represented by the formula (1) and a structural unit represented by the formula (B). In the particle, when a structure obtained by removing R²² from the structural unit represented by the formula (B) is represented by B-X, a content of the B-X in the particle is 5.0 mass % or more and 17.5 mass % or less.

### BACKGROUND OF THE INVENTION

### Field of the Invention

The present invention relates to a particle, a test particle, a reagent, and a test kit, and a detection method.

### Description of the Related Art

In recent years, investigations involving purifying and quantifying a target substance through use of a test particle in which a ligand having an affinity for the target substance and a particle are chemically bonded to each other have been widely made.

In Japanese Patent Application Laid-Open No. 2007-224213, there is a description of a particle having a core-shell structure and containing a carboxy group or a 2,3-dihydroxypropyl group, which is a reactive functional group, ethylene glycol dimethacrylate, which is a hydrophilic crosslinking monomer, and the like in a shell.

In the particle to be used for such purposes, it is preferred that an antibody or an antigen serving as a ligand be chemically bonded uniformly to a particle surface, to thereby increase an agglutination speed to the target substance and improve sensitivity. When chemical bonding to a latex particle is performed, the particle is required to be purified by performing sedimentation of the particle with a centrifugal separator or the like, removal of a supernatant, and subsequent redispersion of a particle sediment in a reactive functional group activation step on the outermost surface. In order to uniformly bond the ligand substance, it is important that the particle be uniformly redispersed in a redispersion step.

In Japanese Patent Application Laid-Open No. 2001-228149, there is a description of a method of controlling surface charge by adding a surfactant or a dispersing aid at the time of preparation of a particle, to thereby enhance redispersibility. However, in the method of controlling surface charge by adding the additive, it becomes difficult to add an antigen (or an antibody) to the particle surface, and the agglutination by an immunoreaction may be inhibited, with the result that complicated and cumbersome adjustments are required depending on the kind of the antigen (or the antibody) to be bonded. Thus, there is a demand for a method of improving the redispersibility of the particle.

### SUMMARY OF THE INVENTION

An object of the present invention is to provide a particle and a test particle improved in redispersibility of the particle, in particular, in redispersibility thereof in a reactive functional group activation step.

A particle according to one aspect of the present invention is a particle including a copolymer having a structural unit represented by the following formula (1) and a structural unit represented by the following formula (B), wherein, when a structure obtained by removing R²² from the structural unit represented by the formula (B) is represented by B-X, a content of the B-X in the particle is 5.0 mass % or more and 17.5 mass % or less:

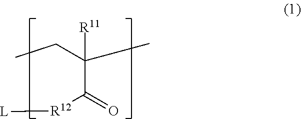

in the formula (1), L represents an unsubstituted or substituted divalent or more and hexavalent or less hydrocarbon group having 1 or more and 15 or less carbon atoms, which may have an oxy group, R¹¹ represents a hydrogen atom or a methyl group, R¹² represents an oxygen atom or an imino group, and R¹¹, R¹², and L may vary depending on each structural unit;

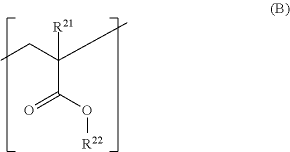

in the formula (B), R²¹ represents a hydrogen atom or a methyl group, R²² represents a group having an epoxy group, a group having a hydroxy group, or a group having a carboxy group or a salt thereof, and R²¹ and R²² may vary depending on each structural unit.

In addition, a particle according to one aspect of the present invention is a particle obtained by a production method including a polymerization step of performing a polymerization reaction of a reaction system containing at least any one of glycidyl methacrylate and glycidyl acrylate, a monomer represented by the following formula (G1), and a monomer represented by the following formula (A1), wherein, in the polymerization step, a total content of the glycidyl methacrylate and the glycidyl acrylate with respect to a content of the monomer represented by the following formula (A1) is 10 mass % or more and 70 mass % or less:

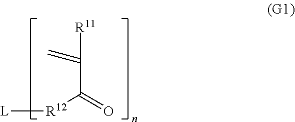

in the formula (G1), L represents an unsubstituted or substituted hydrocarbon group having 1 or more and 15 or less carbon atoms, which may have an oxy group, “n” represents an integer of 2 or more and 6 or less, R¹¹ represents a hydrogen atom or a methyl group, and R¹² represents an oxygen atom or an imino group;

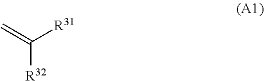

where R³¹ represents a hydrogen atom or a methyl group, R³² represents a substituted or unsubstituted phenyl group or a substituted or unsubstituted naphthyl group, and a substituent thereof is a methyl group, an ethyl group, a hydroxy group, a carboxy group or a salt thereof, or a sulfo group or a salt thereof.

In addition, a particle according to one aspect of the present invention is a particle obtained by a production method including a polymerization step of performing a polymerization reaction of a reaction system containing at least any one of glycidyl methacrylate and glycidyl acrylate and a monomer represented by the following formula (G1), wherein, when a structure obtained by removing R²² from a structural unit represented by the following formula (B) derived from at least any one of the glycidyl methacrylate and the glycidyl acrylate in the particle is represented by B-X, a content of the B-X in the particle is 5.0 mass % or more and 17.5 mass % or less:

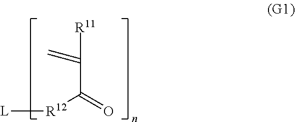

in the formula (G1), L represents an unsubstituted or substituted hydrocarbon group having 1 or more and 15 or less carbon atoms, which may have an oxy group, “n” represents an integer of 2 or more and 6 or less, R¹¹ represents a hydrogen atom or a methyl group, and R¹² represents an oxygen atom or an imino group;

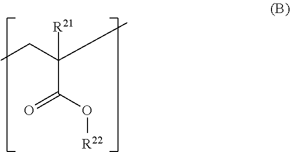

in the formula (B), R²¹ represents a hydrogen atom or a methyl group, R²² represents a group having an epoxy group, a group having a hydroxy group, or a group having a carboxy group or a salt thereof, and R²¹ and R²² may vary depending on each structural unit.

In addition, according to the present invention, there is provided a test particle including a ligand added to a surface of the above-mentioned particle. In addition, according to the present invention, there is provided a reagent including the above-mentioned test particle dispersed in an aqueous solution. In addition, according to the present invention, there is provided a test kit including the above-mentioned reagent and a case configured to enclose the reagent. In addition, according to the present invention, there is provided a method of detecting a target substance in a specimen, the method including mixing the above-mentioned reagent and the specimen that may contain the target substance. In addition, according to the present invention, there is provided a method of detecting a target substance in a specimen by an agglutination method, the method including: mixing the above-mentioned reagent with a specimen that may contain the target substance to provide a mixed liquid; irradiating the mixed liquid with light; and detecting at least any one of transmitted light and scattered light from the light with which the mixed liquid is irradiated.

Further features of the present invention will become apparent from the following description of exemplary embodiments.

### DESCRIPTION OF THE EMBODIMENTS

Embodiments of the present invention are described in detail below, but the technical scope of the present invention is not limited to the embodiments.

### Problems to be Solved by Particle According to Embodiment of the Present Invention

According to the investigations made by the inventors of the present invention, in the configuration of the particle as described in Japanese Patent Application Laid-Open No. 2007-224213, there have been cases in which, when the particle is redispersed by centrifugal precipitation treatment, it takes a long time to redisperse the particle, and in which the particle diameter does not become equal to the initial particle diameter after redispersion, and hence there has been a problem in redispersibility. The inventors of the present invention have found that, in particular, when a ligand is chemically bonded to a particle surface, the particles are liable to be agglutinated in a reactive functional group activation step, which makes redispersion further difficult.

In addition, in Japanese Patent Application Laid-Open No. 2001-228149, there is a description of a method involving using an additive in order to improve the redispersibility, but complicated and cumbersome adjustments are required in some cases depending on the kind of the ligand to be bonded. The details of the particle are not described, and the reactive functional group activation step is also not described.

A particle according to an embodiment of the present invention can solve those problems. The details thereof are described below.

### <Chemical Structure of Particle According to the Present Invention>

The particle according to the present invention is a particle including a copolymer having a structural unit represented by the following formula (1) and a structural unit represented by the following formula (B), in which, when a structure obtained by removing R²² from the structural unit represented by the formula (B) is represented by B-X, a content of the B-X in the particle is 5.0 mass % or more and 17.5 mass % or less.

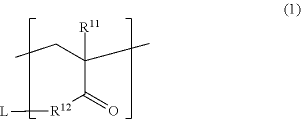

In the formula (1), L represents an unsubstituted or substituted divalent or more and hexavalent or less hydrocarbon group having 1 or more and 15 or less carbon atoms, which may have an oxy group. An example of the substituent thereof is a hydroxy group. Lis bonded to R¹² in each of “n” (“n” represents 2 or more and 6 or less) different structural units. That is, R¹² is bonded to R¹² in another structural unit through L.

R¹¹ represents a hydrogen atom or a methyl group, and R¹² represents an oxygen atom or an imino group.

R¹¹, R¹², and L may vary depending on each structural unit.

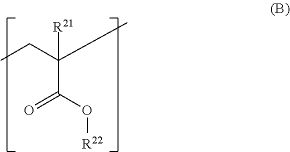

In the formula (B), R²¹ represents a hydrogen atom or a methyl group, R²² represents a group having an epoxy group, a group having a hydroxy group, or a group having a carboxy group or a salt thereof, and R²¹ and R²² may vary depending on each structural unit.

The inventors of the present invention have made extensive investigations, and as a result, have found that it is important that the particle have the structural unit represented by the formula (1), which is a crosslinked structure, and the structural unit represented by the formula (B), which is a hydrophilic structure, and that the amount of the structure (B-X), which exhibits the hydrophilicity of the structural unit represented by the formula (B), be appropriate. The B-X represents the structure obtained by removing R²² from the structural unit represented by the formula (B).

The structural unit represented by the formula (B) has the effect of imparting hydrophilicity and flexibility to the particle, and the structural unit represented by the formula (1) has the effect of imparting hardness to the particle. A specific improvement in redispersibility can only be achieved by combining those characteristics and appropriately adjusting the amounts thereof.

That is, it has been found that the particle according to the present invention has the structural unit represented by the formula (1) and the structural unit represented by the formula (B), and contains the B-X in an amount of 5.0 mass % or more and 17.5 mass % or less, to thereby improve the redispersibility. The particle exhibited a more significant effect, in particular, in the reactive functional group activation step.

It is conceived that, when the structural unit represented by the formula (B), which is a structure exhibiting hydrophilicity, has a crosslinked structure because of the structural unit represented by the formula (1) and contains the B-X in a certain content, both the suppression of hydrophobic interaction and the improvement in particle hardness can be achieved.

The redispersibility was not improved merely by incorporating the structural unit represented by the formula (1), and the effect of improving the redispersibility was not observed even when the content of the B-X was reduced within a certain range. Thus, the inventors of the present invention have found that the above-mentioned range is specifically important.

In the present invention, the copolymer having the structural unit represented by the formula (1) and the structural unit represented by the formula (B) may have the structural unit represented by the formula (1) in which L represents any one of a structure represented by the following formula (L-1), a structure represented by the following formula (L-2), a structure represented by the following formula (L-3), a structure represented by the following formula (L-4), a structure represented by the following formula (L-5), a structure represented by the following formula (L-6), a structure represented by the following formula (L-7), a structure represented by the following formula (L-8), a structure represented by the following formula (L-9), a structure represented by the following formula (L-10), a structure represented by the following formula (L-11), a structure represented by the following formula (L-12), a structure represented by the following formula (L-13), a structure represented by the following formula (L-14), and a structure represented by the following formula (L-15). L may vary depending on each structural unit.

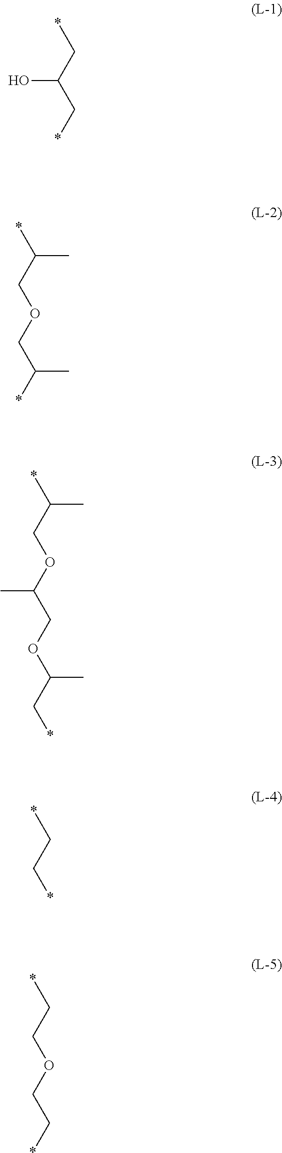

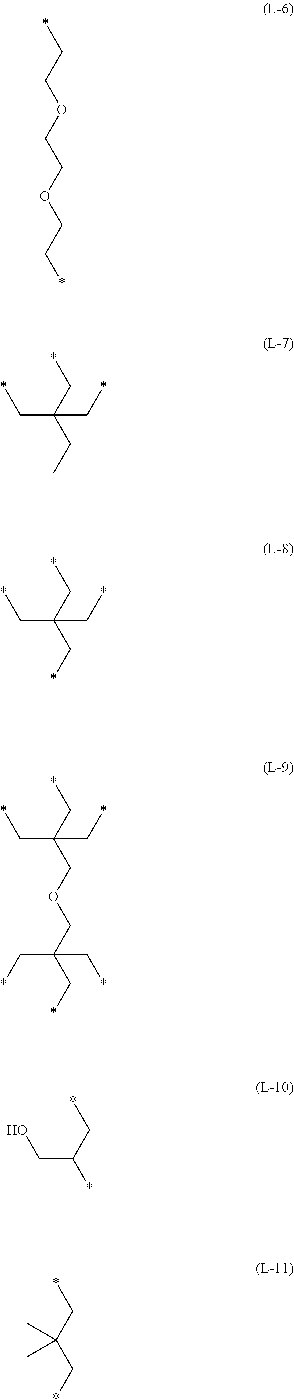

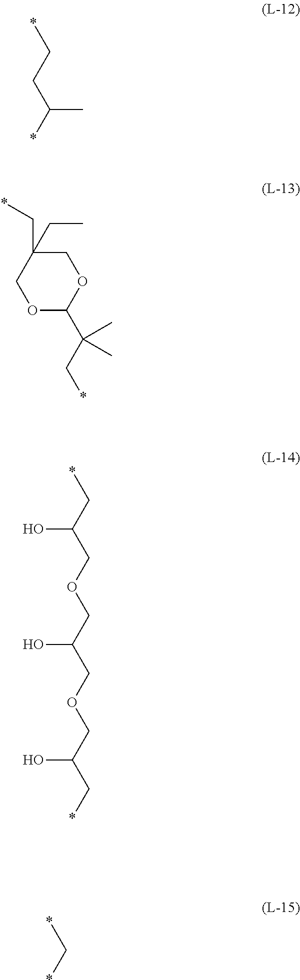

“*” represents a bonding position with R¹² in the formula (1).

The structural unit represented by the formula (1) in the present invention may be any one of a structural unit represented by the following formula (1-1), a structural unit represented by the following formula (1-2), a structural unit represented by the following formula (1-3), a structural unit represented by the following formula (1-4), a structural unit represented by the following formula (1-5), a structural unit represented by the following formula (1-6), a structural unit represented by the following formula (1-7), a structural unit represented by the following formula (1-8), a structural unit represented by the following formula (1-9), a structural unit represented by the following formula (1-10), a structural unit represented by the following formula (1-11), a structural unit represented by the following formula (1-12), a structural unit represented by the following formula (1-13), a structural unit represented by the following formula (1-14), and a structural unit represented by the following formula (1-15), but the structural unit represented by the formula (1) is not limited thereto. The specific structure of the structural unit represented by the formula (1) may vary depending on each structural unit.

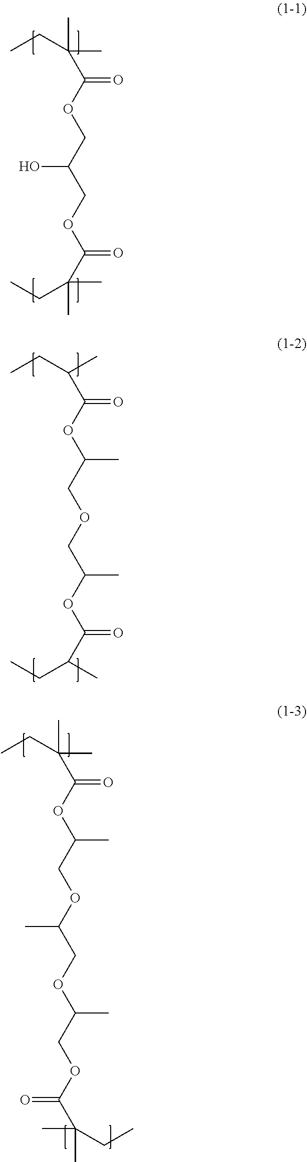

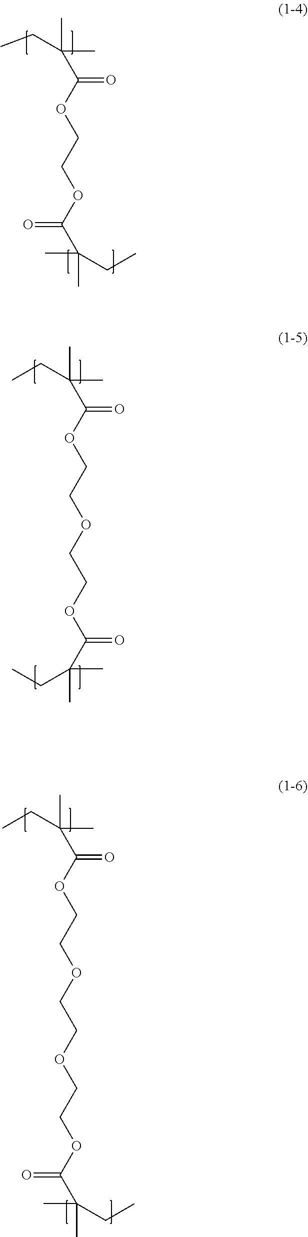

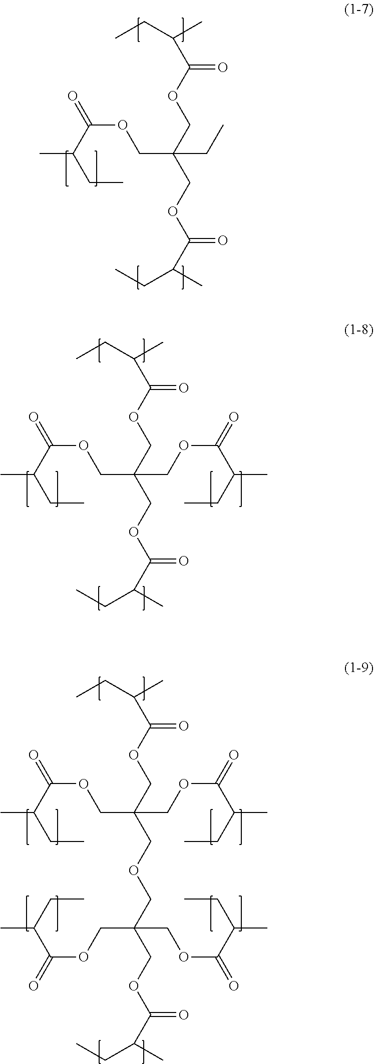

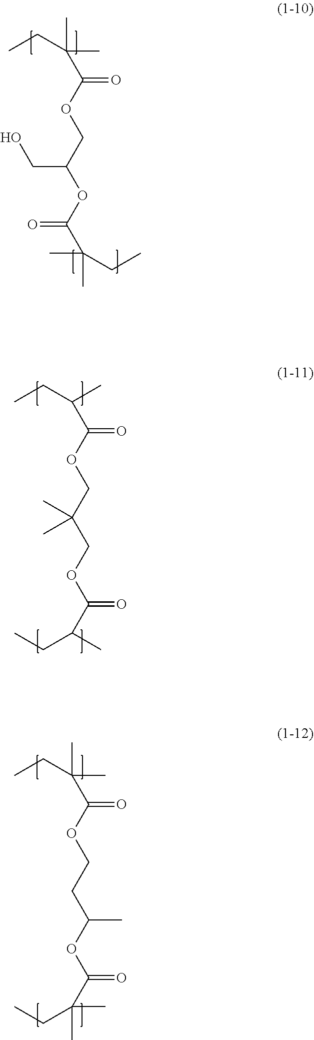

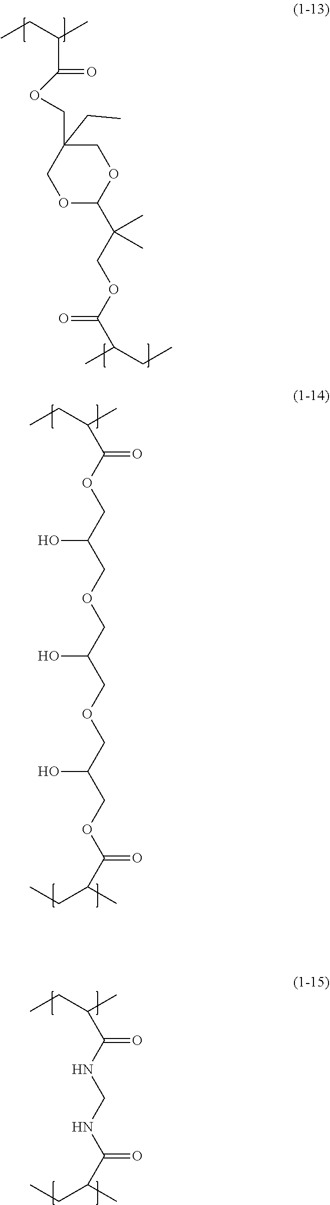

It is preferred that the copolymer having the structural unit represented by the formula (1) and the structural unit represented by the formula (B) have at least any one of the structural unit represented by the formula (1-1) and the structural unit represented by the formula (1-2) out of those structural units as the structural unit represented by the formula (1). The reason for this is conceived as described below. The dual crosslinked structure enables crosslinking to be performed under a state in which the polymer density of the particle is low, and further, the presence of a branched chain in the crosslinked structure reduces the crystalline structure in the crosslinked structure. It is conceived that, with this configuration, the decrease in redispersibility caused by the dense entanglement between polymer chains is prevented, and the hydrophobic interaction of the particles is reduced by the reduction in crystalline structure, leading to an improvement in redispersibility.

The above-mentioned structure may be formed by adding a crosslinking agent monomer at the time of production.

Specific examples of the crosslinking agent monomer include ethylene glycol diacrylate, ethylene glycol dimethacrylate, trimethylolpropane triacrylate, trimethylolpropane trimethacrylate, pentaerythritol triacrylate, pentaerythritol trimethacrylate, dipentaerythritol hexaacrylate, dipentaerythritol hexamethacrylate, neopentyl glycol diacrylate, neopentyl glycol dimethacrylate, 1,3-butanediol diacrylate, 1,3-butanediol dimethacrylate, dioxane glycol diacrylate, dioxane glycol dimethacrylate, glycerin 1,3-diglycerolate diacrylate, glycerin 1,3-diglycerolate dimethacrylate, methylenebisacrylamide, glycerol diacrylate, glycerol dimethacrylate, dipropylene glycol diacrylate, and dipropylene glycol dimethacrylate, but the crosslinking agent monomer is not limited in the range that satisfies the formula (1).

Of those, glycerol dimethacrylate and dipropylene glycol diacrylate are preferred because the structural unit represented by the formula (1-1) and the structural unit represented by the formula (1-2) are obtained.

In addition, it is preferred that the content of the structural unit represented by the formula (1) in the particle be 1 mass % or more and 10 mass % or less.

The effect of improving the hardness of the particle can be enhanced by setting the content to 1 mass % or more, and the effect of suppressing the hydrophobic interaction of the particles can be enhanced by setting the content to 10 mass % or less.

In addition, the particle according to the present invention has the structural unit represented by the formula (B).

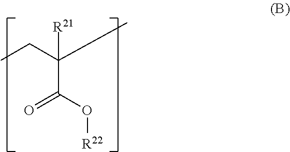

In the formula (B), R²¹ represents a hydrogen atom or a methyl group, R²² represents a group having a hydrogen atom, a group having an epoxy group, a group having a hydroxy group, or a group having a carboxy group or a salt thereof, and R²¹ and R²² may vary depending on each structural unit.

It is preferred that the structural unit represented by the formula (B) have a hydroxy group or a carboxy group. It is more preferred that the structural unit represented by the formula (B) simultaneously have a hydroxy group and a carboxy group.

An example of the structural unit represented by the formula (B) is a structural unit represented by the following formula (B-11).

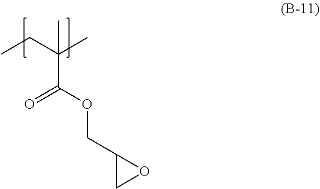

In the present invention, the structural unit represented by the formula (B), for example, the structural unit represented by the formula (B) having an epoxy group is obtained by adding a monomer at the time of production of the particle. The monomer to be added is not particularly limited, but examples thereof include glycidyl methacrylate and glycidyl acrylate.

The structural unit represented by the formula (B) is preferably a structural unit represented by the following formula (B-1).

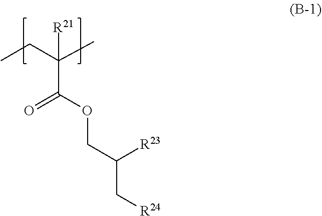

In the formula (B-1), R²¹ represents a hydrogen atom or a methyl group, and at least one of R²³ and R²⁴ represents a hydroxy group, and the other thereof represents a hydroxy group, a structure represented by the following formula (B-2), or a structure represented by the following formula (B-3).

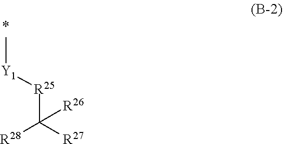

R²⁵ represents a single bond or a methylene group.

R²⁶, R²⁷, and R²⁸ each independently represent a hydrogen atom, an alkyl group having 1 or more and 3 or less carbon atoms, a hydroxy group, a hydroxymethyl group, a carboxy group, an amino group, or a thiol group.

Y₁ represents a sulfur atom or an imino group.

“*” represents a bonding position in the structural unit represented by the formula (B-1).

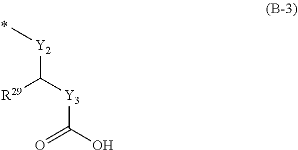

R²⁹ represents a hydrogen atom, an alkyl group having 1 or more and 3 or less carbon atoms, a hydroxy group, a hydroxymethyl group, or a carboxy group.

Y₂ represents a sulfur atom or an imino group.

Y₃ represents a single bond or a methylene group.

“*” represents a bonding position in the structural unit represented by the formula (B-1).

Examples of the structural unit represented by the formula (B-1) having the structure represented by the formula (B-2) or the structure represented by the formula (B-3) include structural units represented by any one of the following formulae (B-12) to (B-34), but the structural unit represented by the formula (B-1) is not limited thereto. The specific structure of the structural unit represented by the formula (B) may vary depending on each structural unit.

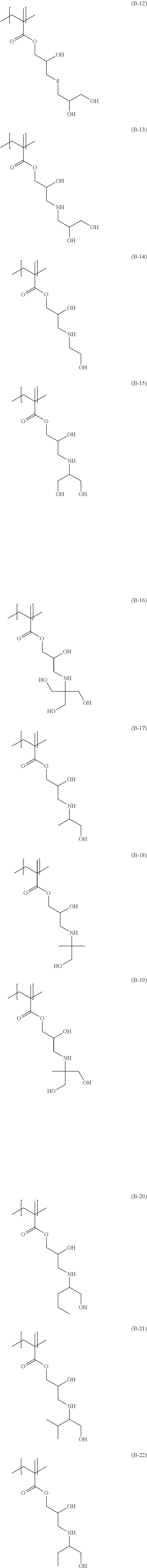

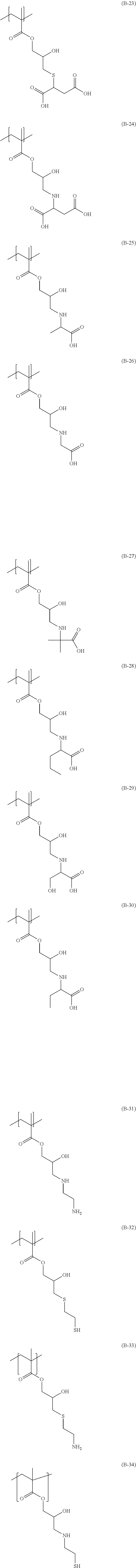

The structure having a hydroxy group, a carboxy group, an amino group, or a thiol group in the formula (B) is obtained by adding a modifier to the particle having an epoxy group introduced thereto. The modifier to be added is not particularly limited, but examples thereof include mercaptosuccinic acid, aspartic acid, 3-mercapto-1,2-propanediol, 3-amino-1,2-propanediol, 2-amino-1,3-propanediol, ethanolamine, trishydroxymethylaminomethane, 1,2-ethylenediamine, 1,2-ethanedithiol, and 2-aminoethanethiol.

In the present invention, it is preferred that the particle further have a structural unit represented by the following formula (A). When the particle has the structural unit represented by the formula (A) therein, the refractive index of the particle is increased. When a test reagent reacts with a target substance to be agglutinated, the absorbance is increased. When the particle having a high refractive index is agglutinated, the absorbance is further increased, and hence the difference in absorbance before and after the agglutination is also further increased, resulting in an improvement in sensitivity.

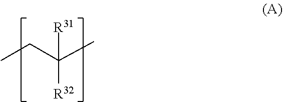

R³¹ represents a hydrogen atom or a methyl group, R³² represents a substituted or unsubstituted phenyl group, or a substituted or unsubstituted naphthyl group, and the substituent thereof is a methyl group, an ethyl group, a hydroxy group, a carboxy group or a salt thereof, a sulfo group or a salt thereof, or the like, and R³¹ and R³² may vary depending on each structural unit.

R³¹ preferably represents a hydrogen atom, and R³² preferably represents a phenyl group.

In the present invention, the structural unit represented by the formula (A) may be formed by adding a monomer at the time of production of the particle.

Specific examples of the monomer include styrenes, 1-vinylnaphthalene, and 2-vinylnaphthalene. Of those, styrene is particularly preferred. Those monomers may be used alone or in combination thereof.

Examples of the styrenes include styrene, α-methylstyrene, o-methylstyrene, m-methylstyrene, p-methylstyrene, 2,4-dimethylstyrene, o-ethylstyrene, m-ethylstyrene, p-ethylstyrene, 4-vinylphenol, 4-vinylbenzoic acid, and sodium p-styrenesulfonate.

In the present invention, when the content of the structural unit represented by the formula (A) in the particle is represented by M(A), and the content of the B-X in the particle is represented by M(B), a value of a mass ratio M(B)/M(A) between the contents is preferably 0.05 or more and 1.00 or less. In addition, the value of M(B)/M(A) is more preferably 0.05 or more and 0.45 or less. When the value of M(B)/M(A) is 0.05 or more, the hydrophilicity can be improved, and the redispersibility can be improved. In addition, when the value of M(B)/M(A) is 1.00 or less, the particle becomes hard, and hence is less liable to be influenced by deformation and the like in centrifugation and redispersion steps. Thus, the redispersibility can be improved.

The volume-average particle diameter of the particle of present invention is not particularly limited, but may be set to 10 nm or more and 1,000 nm or less. In addition, a value of a ratio (Dv/Dn) between a volume-average particle diameter (Dv) and a number-average particle diameter (Dn) is preferably 1.25 or less. Further, the value of Dv/Dn is more preferably less than 1.15. It is known that, as the value of Dv/Dn becomes closer to 1, the particle size distribution is reduced. It is conceived that, when the value of Dv/Dn is set to 1.25 or less, the variation in size of the particles is decreased to reduce the contact area between the particles, to thereby improve the redispersibility.

The particle according to the present invention may be a particle obtained by a production method including a polymerization step of performing a polymerization reaction of a reaction system containing at least any one of glycidyl methacrylate and glycidyl acrylate, a monomer represented by the following formula (G1), and a monomer represented by the following formula (A1), in which, in the polymerization step, a total content of the glycidyl methacrylate and the glycidyl acrylate with respect to a content of the monomer represented by the following formula (A1) is 10 mass % or more and 70 mass % or less.

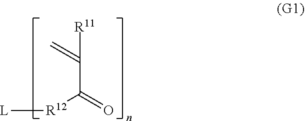

In the formula (G1), L represents an unsubstituted or substituted hydrocarbon group having 1 or more and 15 or less carbon atoms, which may have an oxy group, “n” represents an integer of 2 or more and 6 or less, R¹¹ represents a hydrogen atom or a methyl group, and R¹² represents an oxygen atom or an imino group.

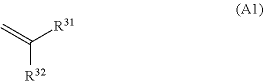

R³¹ represents a hydrogen atom or a methyl group, R³² represents a substituted or unsubstituted phenyl group, or a substituted or unsubstituted naphthyl group, and the substituent thereof is a methyl group, an ethyl group, a hydroxy group, a carboxy group or a salt thereof, or a sulfo group or a salt thereof.

In addition, the particle according to the present invention may be a particle obtained by a production method including a polymerization step of performing a polymerization reaction of a reaction system containing at least any one of glycidyl methacrylate and glycidyl acrylate and the monomer represented by the formula (G1), in which, when a structure obtained by removing R²² from the structural unit represented by the formula (B) derived from at least any one of the glycidyl methacrylate and the glycidyl acrylate in the particle is represented by B-X, a content of the B-X in the particle is 5.0 mass % or more and 17.5 mass % or less. In this case, when the reaction system contains both glycidyl methacrylate and glycidyl acrylate, the structural units represented by the formula (B) include both the structural unit derived from glycidyl methacrylate and the structural unit derived from glycidyl acrylate.

The content of the B-X and the content of each of the structural units in the particle may be calculated from the amounts of monomers loaded in the polymerization step. For example, the content of the B-X may be calculated by calculating the mass of a monomer having the B-X with respect to the total mass of the monomers and the like except a loaded solvent, and further estimating the mass of the B-X. For example, the content of the B-X or the like may be determined by analyzing the solution after the polymerization reaction by gas chromatography to quantify the remaining monomers, and determining a value (polymerization conversion ratio) indicating to which degree the monomers have been converted by polymerization in the polymerization reaction from the amounts of the remaining monomers. In this case, when the amounts of the remaining monomers are equal to or less than the detection limit, the polymerization conversion ratio may be defined to be substantially 100% (all the monomers have been polymerized).

In addition, the particle according to the present invention is preferably a particle, characterized in that the polymerization step is performed by using a water-soluble polymerization initiator. It is preferred that the water-soluble polymerization initiator have an amide structure, an amidine group, or an imidazoline structure.

When the particle produced by the above-mentioned production method is used, there can be provided a particle excellent in redispersibility, in particular, a particle and a test particle improved in redispersibility in the reactive functional group activation step in the case of chemically bonding a ligand to the particle surface. A method of producing a particle is specifically described below.

### <Method of Producing Particle According to the Present Invention>

A method of producing a particle according to the present invention includes a polymerization step of performing a polymerization reaction of a reaction system containing at least any one of glycidyl methacrylate and glycidyl acrylate and the monomer represented by the formula (G1). In the polymerization step, the reaction system may contain the monomer represented by the formula (A1).

In the polymerization step, when the reaction system contains the monomer represented by the formula (A1), the total content of glycidyl methacrylate and glycidyl acrylate with respect to the content of the monomer represented by the formula (A1) may be set to 10 mass % or more and 70 mass % or less.

In the polymerization step, glycidyl methacrylate and glycidyl acrylate, and the polymerization initiator may be partially added first to initiate the polymerization reaction, and then the remaining portions may be added together with the monomer represented by the formula (G1) to continue the polymerization reaction.

As the method of producing a particle, a method involving performing soap-free emulsion polymerization in the polymerization step is preferred. When the soap-free emulsion polymerization is used, the particle size distribution becomes uniform, and the sensitivity becomes stable, thereby being capable of improving the detection limit in a region in which the concentration of a target substance is low.

The polymerization initiator to be used in the production of a particle is not particularly limited, but it is preferred that a water-soluble polymerization initiator be added in the polymerization step. The details of the mechanism are not clear, but are conceived as described below.

It is assumed that, when the water-soluble polymerization initiator is used, the reaction point of the polymerization can be set to the particle surface. It is conceived that, when the monomer represented by the formula (G1) for forming the structural unit represented by the formula (1), that is, a crosslinking agent monomer and the water-soluble polymerization initiator are used together, the hardness of the particle surface can be further improved, and as a result, the redispersibility is improved.

Preferred specific examples of the water-soluble polymerization initiator include 4,4′-azobis(4-cyanovaleric acid), 2,2′-azobis[2-methyl-N-(2-hydroxyethyl) propionamide], 2,2′-azobis[N-(2-carboxyethyl)-2-methylpropionamidine]tetrahydrate, 2,2′-azobis(2-methylpropionamidine) dihydrochloride, 2,2′-azobis[2-(2-imidazolin-2-yl)propane]dihydrochloride, 2,2′-azobis[2-(2-imidazolin-2-yl)propane]disulfate dihydrate, potassium peroxodisulfate, sodium peroxodisulfate, and ammonium peroxodisulfate.

It is preferred that the particle according to the present invention include a compound having an amidine group, an amide structure, or an imidazoline structure. It is preferred that a polymerization initiator having an amide structure, an amidine group, or an imidazoline structure be used in the polymerization step and that the particle have a structure derived from the polymerization initiator. It is more preferred that the particle include the compound having an amidine group, in particular.

In addition, the method of producing a particle may include an addition reaction step of causing an addition reaction with respect to the particle through use of a modifier having a hydroxy group, or a carboxy group or a salt thereof. The above-mentioned modifier for introducing the structure having a hydroxy group, a carboxy group, an amino group, or a thiol group in the structural unit represented by the formula (B) in the particle may be used as the modifier having a hydroxy group, or a carboxy group or a salt thereof. In order to determine whether or not each of the above-mentioned structures has been added to the particle, the reaction solution after the addition reaction may be analyzed by high-performance liquid chromatography. The remaining modifiers are quantified, and a value (addition reaction conversion ratio) indicating to which degree the reaction has progressed through the addition reaction may be evaluated from the amounts of the remaining modifiers. When the amounts of the remaining modifiers are equal to or less than the detection limit, it can be determined that the addition reaction conversion ratio is substantially 100% (all the modifiers have been subjected to the addition reaction).

### <Test Method Using Particle According to the Present Invention>

Although there is no particular limitation on a test method using the particle according to this embodiment, a test method using an antibody (antigen) as a ligand and using an antigen (antibody) as a target substance is preferred. Specific examples thereof include immunochromatography, an immunofluorescence analysis method, chemiluminescence immunoassay, and an agglutination method (so-called latex agglutination method), and the particle may be preferably applied to the agglutination method, which has been widely utilized in fields, such as a clinical test and biochemical research, out of those methods.

In the test method in the agglutination method, for example, a test particle in which an antibody (antigen) is added as a ligand to the surface of the particle according to the present invention may be used. At the time of testing, the above-mentioned test particle is dispersed in an aqueous solution, and the resultant may be used as a reagent. In the method of detecting a target substance in a specimen, the above-mentioned reagent and a specimen that may contain a target substance are mixed with each other. The mixing of the reagent containing the test particle according to this embodiment and the specimen is preferably performed in the range of from pH 3.0 to pH 11.0. In addition, the mixing temperature is in the range of from 20° C. to 50° C., and the mixing time is in the range of from 1 minute to 20 minutes. In addition, it is preferred that a solvent be used in the detection method.

In addition, the concentration of the test particle according to this embodiment in the detection method according to this embodiment is preferably from 0.001 mass % to 5 mass %, more preferably from 0.01 mass % to 1 mass % in the reaction system. The detection method according to this embodiment preferably involves optically detecting an agglutination reaction that occurs as a result of the mixing of the test particle according to this embodiment and the specimen, that is, detecting a target substance in a specimen by an agglutination method. Specifically, the detection method includes: mixing a specimen that may contain a target substance with a reagent containing a test particle to provide a mixed liquid; irradiating the mixed liquid with light; and detecting at least any one of transmitted light and scattered light from the light with which the mixed liquid is irradiated.

When the above-mentioned agglutination reaction that occurs in the mixed liquid is optically detected, the target substance in the specimen can be detected, and further, the concentration of the target substance can also be measured. As a method of optically detecting the agglutination reaction, it is only required that an optical device capable of detecting a scattered light intensity, a transmitted light intensity, an absorbance, and the like be used to measure a change amount of each of these values.

### <Test Reagent for In Vitro Diagnosis>

A test reagent for in vitro diagnosis according to this embodiment, that is, a reagent for use in detection of a target substance in a specimen by in vitro diagnosis, contains the test particle according to this embodiment and a dispersion medium that disperses the test particle. The amount of the test particle according to this embodiment contained in the reagent according to this embodiment is preferably from 0.001 mass % to 20 mass %, more preferably from 0.01 mass % to 10 mass %. The reagent according to this embodiment may contain a third substance, such as a solvent or a blocking agent, in addition to the test particle according to this embodiment to the extent that the object of the present invention can be achieved. The third substance, such as a solvent or a blocking agent, may be incorporated in combination of two or more kinds thereof. Examples of the dispersion medium to be used in the present invention include various buffers, such as a phosphate buffer, a glycine buffer, a Good's buffer, a Tris buffer, and an ammonia buffer, but the dispersion medium contained in the reagent according to this embodiment is not limited thereto.

### <Test Kit>

A kit for use in detection of a target substance in a specimen by in vitro diagnosis according to this embodiment includes the above-mentioned reagent and a case that encloses the reagent.

It is preferred that the kit according to this embodiment further include a reaction buffer containing albumin in addition to the reagent according to this embodiment.

An example of albumin is serum albumin, and protease-treated albumin may also be used. The amount of albumin contained in the reaction buffer containing albumin is generally from 0.001 mass % to 5 mass %, but the kit according to this embodiment is not limited thereto. A sensitizer for the agglutination method may be incorporated into both the reagent according to this embodiment and the reaction buffer containing albumin or any one of the reagent according to this embodiment or the reaction buffer containing albumin. Examples of the sensitizer for the agglutination method include polyvinyl alcohol, polyvinyl pyrrolidone, and polyalginic acid, but the present invention is not limited thereto. In addition, the kit according to this embodiment may include a positive control, a negative control, a serum diluent, and the like in addition to the reagent according to this embodiment and the reaction buffer containing albumin. In addition to serum or physiological saline free of the target substance that may be subjected to the assay, a solvent may be used as a medium for the positive control or the negative control. The kit according to this embodiment may be used in a method of detecting a target substance according to this embodiment as in a typical kit for use in detection of a target substance in a specimen by in vitro diagnosis. In addition, the concentration of the target substance can be measured by a hitherto known method, and the method is suitably used in the detection of the target substance in the specimen by, in particular, an agglutination method.

### <Method of Producing Test Particle>

In order to produce a test particle, the particle according to this embodiment is redispersed. Examples of a method of redispersing the particle include: a redispersion method by stirring; and a redispersion method by vibration. Examples of the redispersion method by stirring include: a stirring method involving forming a vortex with a vortex mixer or the like; and a method involving directly stirring a precipitate. An example of the redispersion method by vibration is an ultrasonic dispersion method. Examples of a dispersion medium include: various buffers, such as a phosphate buffer, a glycine buffer, a Good's buffer, a Tris buffer, and an ammonia buffer.

The particle according to the present invention may be used as a test particle with respect to a specific target by adding a ligand to the surface of the particle according to the present invention. When the ligand is added to the surface by chemical bonding, it is required to activate the reactive functional groups on the particle surface. This step is referred to as “reactive functional group activation step.” A method to be used in the reactive functional group activation step is not particularly limited, and a general method may be used.

When the reactive functional group is a carboxy group, the reactive functional group may be activated through use of a condensing agent and a condensing aid. The condensing agent is preferably 1-(3-dimethylaminopropyl)-3-ethylcarbodiimide hydrochloride, N,N′-dicyclohexylcarbodiimide, or N,N′-diisopropylcarbodiimide, and the condensing aid is preferably 1-hydroxybenzotriazole or sodium N-hydroxysulfosuccinimide.

In addition, the ligand in the present invention may be defined to be a compound that is specifically bonded to a receptor that a specific target substance has. The site at which the ligand is bonded to the target substance is predetermined, and the ligand has a selectively or specifically high affinity for the target substance. Examples of the ligand include: an antigen and an antibody; an enzyme protein and a substrate thereof; a signal substance, such as a hormone or a neurotransmitter, and a receptor thereof; and a nucleic acid. However, the ligand in the present invention is not limited thereto. An example of the nucleic acid is deoxyribonucleic acid. The test particle according to the present invention has a selectively or specifically high affinity for the target substance. The ligand in the present invention is preferably any one of an antibody, an antigen, or a nucleic acid.

The test particle using the particle according to the present invention may be obtained as described above.

### EXAMPLES

The present invention is described in detail below by way of Examples. However, the present invention is not limited to these Examples.

### [Production Example 1] (Synthesis of Particle 1)

The following materials were weighed in a 2 L four-necked separable flask to provide a mixed liquid.
33.85 g of styrene (St: Kishida Chemical Co., Ltd.)
0.61 g of divinylbenzene (DVB: Kishida Chemical Co., Ltd.)
14.79 g of pre-addition glycidyl methacrylate (GMA: Kishida Chemical Co., Ltd.)
1,195.00 g of ion-exchanged water

After that, the mixed liquid was held at 70° C. while being stirred at 100 rpm, and nitrogen was flowed at a flow rate of 200 ml/min to remove oxygen from the inside of the four-necked separable flask. Next, a separately prepared dissolved liquid, which had been obtained by dissolving 1.67 g of pre-addition V-50 (FUJIFILM Wako Pure Chemical Corporation) in 30.00 g of ion-exchanged water, was added to the mixed liquid to initiate soap-free emulsion polymerization. Two hours after the initiation of the polymerization, 2.82 g of post-addition GMA and 1.85 g of glycerol dimethacrylate (GDM: manufactured by Tokyo Chemical Industry Co., Ltd.) were added to a radical polymerization reaction field. Further, a separately prepared dissolved liquid, which had been obtained by dissolving 0.06 g of post-addition V-50 in 10.00 g of ion-exchanged water, was added. The mixture was held at 70° C. while being stirred at 100 rpm for 46 hours, and was then slowly cooled to room temperature. At this point, the contents of the 2 L flask were sampled and analyzed by gas chromatography. The remaining monomers were quantified, and the value (polymerization conversion ratio) indicating to which degree the monomers had been converted by polymerization in the polymerization reaction was evaluated from the amounts of the remaining monomers. As a result, it was recognized that the amounts of the remaining monomers were equal to or less than the detection limit and that the polymerization conversion ratio was substantially 100%.

Next, an addition reaction was performed on particles. An aqueous solution in which mercaptosuccinic acid (MSA: FUJIFILM Wako Pure Chemical Corporation) and 3-mercapto-1,2-propanediol (MPD: FUJIFILM Wako Pure Chemical Corporation) were previously mixed at a ratio of 1:1 in terms of molar equivalent and dissolved was prepared. This aqueous solution was added so that the total molar amount of the MSA and the MPD became the same as the molar amount of the GMA, and triethylamine (Kishida Chemical Co., Ltd.) was added to adjust the pH to 10. Next, the resultant was increased in temperature to 70° C. while being stirred at 200 rpm, and was further held in this state for 18 hours to provide a dispersion of particles 1. The reaction solution after the addition reaction was analyzed by high-performance liquid chromatography. The amounts of the remaining modifiers (MSA and MPD in Production Example 1) were determined, and a value (addition reaction conversion ratio) indicating to which degree the reaction had progressed through the addition reaction was evaluated from the amounts of the remaining modifiers. As a result, it was recognized that the amounts of the remaining modifiers were equal or less than the detection limit and that the addition reaction conversion ratio was substantially 100%. The particles 1 were separated from the dispersion with a centrifugal separator, and the particles 1 were further redispersed in ion-exchanged water; the operation was repeated five times to purify the particles 1, which were stored in the state of an aqueous dispersion in which the concentration of the particles 1 was finally adjusted to 1.0 mass %.

The results obtained by evaluating particle physical properties of the particles 1 and the mass ratio of the B-X in the particles 1 are shown in Table 1.

### [Production Example 2] (Synthesis of Particle 2)

A dispersion of particles 2 was obtained by the same experimental operation as in Production Example 1 except that 1.97 g of dipropylene glycol diacrylate (DPGDA: manufactured by Tokyo Chemical Industry Co., Ltd.) was used instead of 1.85 g of the GDM. The polymerization conversion ratio was evaluated by gas chromatography, and the addition reaction conversion ratio was evaluated by high-performance liquid chromatography. As a result, it was recognized that both the ratios were substantially 100%.

The results obtained by evaluating particle physical properties of the particles 2 and the mass ratio of the B-X in the particles 2 are shown in Table 1.

### [Production Example 3] (Synthesis of Particle 3)

A dispersion of particles 3 was obtained by the same experimental operation as in Production Example 1 except that the DVB was not added. The polymerization conversion ratio was evaluated by gas chromatography, and the addition reaction conversion ratio was evaluated by high-performance liquid chromatography. As a result, it was recognized that both the ratios were substantially 100%.

The results obtained by evaluating particle physical properties of the particles 3 and the mass ratio of the B-X in the particles 3 are shown in Table 1.

### [Production Example 4] (Synthesis of Particle 4)

A dispersion of particles 4 was obtained by the same experimental operation as in Production Example 1 except that: the amount of the pre-addition GMA was changed from 14.79 g to 3.13 g; the amount of the pre-addition V-50 was changed from 1.67 g to 1.27 g; the amount of the post-addition GMA was changed from 2.82 g to 0.60 g; 1.61 g of ethylene glycol dimethacrylate (EDMA: manufactured by Tokyo Chemical Industry Co., Ltd.) was used instead of 1.85 g of the GDM; and the amount of the post-addition V-50 was changed from 0.06 g to 0.01 g. The polymerization conversion ratio was evaluated by gas chromatography, and the addition reaction conversion ratio was evaluated by high-performance liquid chromatography. As a result, it was recognized that both the ratios were substantially 100%.

The results obtained by evaluating particle physical properties of the particles 4 and the mass ratio of the B-X in the particles 4 are shown in Table 1.

### [Production Example 5] (Synthesis of Particle 5)

A dispersion of particles 5 was obtained by the same experimental operation as in Production Example 1 except that 1.85 g of the GDM was changed to 1.61 g of the EDMA. The polymerization conversion ratio was evaluated by gas chromatography, and the addition reaction conversion ratio was evaluated by high-performance liquid chromatography. As a result, it was recognized that both the ratios were substantially 100%.

The results obtained by evaluating particle physical properties of the particles 5 and the mass ratio of the B-X in the particles 5 are shown in Table 1.

### [Production Example 6] (Synthesis of Particle 6)

A dispersion of particles 6 was obtained by the same experimental operation as in Production Example 5 except that the amount of the EDMA was changed from 1.61 g to 6.44 g. The polymerization conversion ratio was evaluated by gas chromatography, and the addition reaction conversion ratio was evaluated by high-performance liquid chromatography. As a result, it was recognized that both the ratios were substantially 100%.

The results obtained by evaluating particle physical properties of the particles 6 and the mass ratio of the B-X in the particles 6 are shown in Table 1.

### [Production Example 7] (Synthesis of Particle 7)

A dispersion of particles 7 was obtained by the same experimental operation as in Production Example 1 except that: the amount of the pre-addition GMA was changed from 14.79 g to 19.68 g; the amount of the pre-addition V-50 was changed from 1.67 g to 1.84 g; the amount of the post-addition GMA was changed from 2.82 g to 3.75 g; 1.61 g of the EDMA was used instead of 1.85 g of the GDM; and the amount of the post-addition V-50 was changed from 0.06 g to 0.09 g. The polymerization conversion ratio was evaluated by gas chromatography, and the addition reaction conversion ratio was evaluated by high-performance liquid chromatography. As a result, it was recognized that both the ratios were substantially 100%.

The results obtained by evaluating particle physical properties of the particles 7 and the mass ratio of the B-X in the particles 7 are shown in Table 1.

### [Production Example 8] (Synthesis of Particle 8)

A dispersion of particles 8 was obtained by the same experimental operation as in Production Example 5 except that the amount of the EDMA was changed from 1.61 g to 0.32 g. The polymerization conversion ratio was evaluated by gas chromatography, and the addition reaction conversion ratio was evaluated by high-performance liquid chromatography. As a result, it was recognized that both the ratios were substantially 100%.

The results obtained by evaluating particle physical properties of the particles 8 and the mass ratio of the B-X in the particles 8 are shown in Table 1.

### [Production Example 9] (Synthesis of Particle 9)

A dispersion of particles 9 was obtained by the same experimental operation as in Production Example 5 except that the amount of the EDMA was changed from 1.61 g to 9.66 g. The polymerization conversion ratio was evaluated by gas chromatography, and the addition reaction conversion ratio was evaluated by high-performance liquid chromatography. As a result, it was recognized that both the ratios were substantially 100%.

The results obtained by evaluating particle physical properties of the particles 9 and the mass ratio of the B-X in the particles 9 are shown in Table 1.

### [Production Example 10] (Synthesis of Particle 10)

A dispersion of particles 10 was obtained by the same experimental operation as in Production Example 5 except that the reagents in the addition reaction with respect to the particles were changed from the MSA and the MPD to MSA and 3-amino-1,2-propanediol (APD: manufactured by Tokyo Chemical Industry Co., Ltd.). The polymerization conversion ratio was evaluated by gas chromatography, and the addition reaction conversion ratio was evaluated by high-performance liquid chromatography. As a result, it was recognized that both the ratios were substantially 100%.

The results obtained by evaluating particle physical properties of the particles 10 and the mass ratio of the B-X in the particles 10 are shown in Table 1.

### [Comparative Production Example 1] (Synthesis of Comparative Particle 1)

A dispersion of comparative particles 1 was obtained by the same experimental operation as in Production Example 5 except that: the amount of the pre-addition GMA was changed from 14.79 g to 22.75 g; the amount of the pre-addition V-50 was changed from 1.67 g to 1.94 g; the amount of the post-addition GMA was changed from 2.82 g to 4.33 g; and the amount of the post-addition V-50 was changed from 0.06 g to 0.10 g. The polymerization conversion ratio was evaluated by gas chromatography, and the addition reaction conversion ratio was evaluated by high-performance liquid chromatography. As a result, it was recognized that both the ratios were substantially 100%. The results obtained by evaluating particle physical properties of the comparative particles 1 and the mass ratio of the B-X in the comparative particles 1 are shown in Table 1.

### [Comparative Production Example 2] (Synthesis of Comparative Particle 2)

A dispersion of comparative particles 2 was obtained by the same experimental operation as in Production Example 5 except that: the amount of the pre-addition GMA was changed from 14.79 g to 2.27 g; the amount of the pre-addition V-50 was changed from 1.67 g to 1.25 g; the amount of the post-addition GMA was changed from 2.82 g to 0.43 g; and the amount of the post-addition V-50 was changed from 0.06 g to 0.01 g. The polymerization conversion ratio was evaluated by gas chromatography, and the addition reaction conversion ratio was evaluated by high-performance liquid chromatography. As a result, it was recognized that both the ratios were substantially 100%. The results obtained by evaluating particle physical properties of the comparative particles 2 and the mass ratio of the formula B-X in the comparative particles 2 are shown in Table 1.

### [Comparative Production Example 3] (Synthesis of Comparative Particle 3)

A dispersion of comparative particles 3 was obtained by the same experimental operation as in Production Example 5 except that: the amount of the pre-addition GMA was changed from 14.79 g to 54.02 g; the amount of the pre-addition V-50 was changed from 1.67 g to 3.00 g; the amount of the post-addition GMA was changed from 2.82 g to 10.29 g; and the amount of the post-addition V-50 was changed from 0.06 g to 0.23 g. The polymerization conversion ratio was evaluated by gas chromatography, and the addition reaction conversion ratio was evaluated by high-performance liquid chromatography. As a result, it was recognized that both the ratios were substantially 100%. The results obtained by evaluating particle physical properties of the comparative particles 3 and the mass ratio of the B-X in the comparative particles 3 are shown in Table 1.

### [Comparative Production Example 4] (Synthesis of Comparative Particle 4)

1,195.00 g of ion-exchanged water and 0.23 g of an organic solvent (Shellsol TK: manufactured by Shell Chemicals Japan Ltd.) were weighed in a 2 L four-necked separable flask to provide a mixed liquid. After that, the mixed liquid was held at 70° C. while being stirred at 100 rpm, and nitrogen was flowed at a flow rate of 200 ml/min to remove oxygen from the inside of the four-necked separable flask. Next, a separately prepared mixed liquid of 33.85 g of St, 0.61 g of DVB, 27.08 g of GMA, and 1.67 g of azobisisobutyronitrile (AIBN: FUJIFILM Wako Pure Chemical Corporation) was added to the above-mentioned mixed liquid to initiate emulsion polymerization. The resultant was held at 70° C. while being stirred at 100 rpm for 46 hours, and was then slowly cooled to room temperature. At this point, the contents of the 2 L flask were sampled, and the radical polymerization conversion ratio was evaluated by gas chromatography. As a result, it was recognized that the radical polymerization conversion ratio was substantially 100%.

Next, the addition reaction with respect to particles was performed in the same manner as in Production Example 1.

The results obtained by evaluating particle physical properties of the comparative particles 4 and the mass ratio of the formula (B-X) in the comparative particles 4 are shown in Table 1.

TABLE 1

| | | | (Content of B-X)/ | Monomer for | |
|---|---|---|---|---|---|
| | | | (content of structural | forming | Content of |
| | | | unit represented | structure | structural unit |
| | | Content | by formula (A)) | represented by | represented by |
| | Particle | of B-X | (M(B)/M(A)) | formula (1) | formula (1) |
| Production | Particle 1 | 15.1% | 0.32 | GDM | 2.6% |
| Example 1 | | | | | |
| Production | Particle 2 | 15.0% | 0.32 | DPGDA | 2.7% |
| Example 2 | | | | | |
| Production | Particle 3 | 15.2% | 0.32 | GDM | 2.6% |
| Example 3 | | | | | |
| Production | Particle 4 | 5.1% | 0.07 | EDMA | 3.6% |
| Example 4 | | | | | |
| Production | Particle 5 | 15.1% | 0.32 | EDMA | 2.3% |
| Example 5 | | | | | |
| Production | Particle 6 | 14.1% | 0.32 | EDMA | 8.5% |
| Example 6 | | | | | |
| Production | Particle 7 | 17.4% | 0.42 | EDMA | 1.9% |
| Example 7 | | | | | |
| Production | Particle 8 | 15.4% | 0.32 | EDMA | 0.5% |
| Example 8 | | | | | |
| Production | Particle 9 | 13.6% | 0.32 | EDMA | 12.2% |
| Example 9 | | | | | |
| Production | Particle 10 | 15.3% | 0.32 | EDMA | 2.3% |
| Example 10 | | | | | |
| Comparative | Comparative | 18.5% | 0.49 | EDMA | 1.8% |
| Production | Particle 1 | | | | |
| Example 1 | | | | | |
| Comparative | Comparative | 3.9% | 0.05 | EDMA | 3.8% |
| Production | Particle 2 | | | | |
| Example 2 | | | | | |
| Comparative | Comparative | 24.3% | 1.16 | EDMA | 1.0% |
| Production | Particle 3 | | | | |
| Example 3 | | | | | |
| Comparative | Comparative | 18.5% | 0.49 | Absent | 0.0% |
| Production | Particle 4 | | | | |
| Example 4 | | | | | |

### [Evaluation 1] Evaluation of Redispersibility of Activated Particle (Reactive Functional Group Activation Step of Particle)

180 μL of a 1.7 mass % water suspension of the particles produced in each of Production Examples 1 to 10 and Comparative Production Examples 1 to 4 was dispensed in a 1.5 mL microtube. The following materials were added thereto.
90 μL of a 5.0% aqueous solution of 1-(3-dimethylaminopropyl)-3-ethylcarbodiimide hydrochloride serving as a condensing agent
90 μL of a 5.0% aqueous solution of sodium N-hydroxysulfosuccinimide serving as a condensing aid

The mixture was stirred at room temperature for 30 minutes to provide particles that were activated (hereinafter referred to as “activated particles”).

### (Evaluation of Redispersibility)

A dispersion of the activated particles produced above was sedimented at a force of 20,000 G for 5 minutes with a centrifugal separator. The supernatant was drained, and 250 μL of a Tris-HCl buffer having a pH of 8.0 was newly added.

Six microtubes each containing the activated particle sediment having the buffer added thereto were prepared, and ultrasonic dispersion treatment was performed 20 times with an ultrasonic disintegrator Bioruptor II (TYPE 6) manufactured by Sonicbio Co., Ltd. in a High mode at a liquid temperature of 4° C. for 16 seconds.

The state of each of the particles in the microtubes after the ultrasonic dispersion treatment was visually checked. After that, the particle diameter of each of the particles was measured with ZETASIZER ULTRA manufactured by Spectris Co., Ltd., and an average value of changes in particle diameter before and after the activation in the six microtubes was calculated. The resultant average value was evaluated as described below.
A: (Particle diameter of activated particles after ultrasonic dispersion)/(particle diameter of particles before activation)=1.05 or less
B: (Particle diameter of activated particles after ultrasonic dispersion)/(particle diameter of particles before activation)=more than 1.05 and 1.1 or less
C: (Particle diameter of activated particles after ultrasonic dispersion)/(particle diameter of particles before activation)=more than 1.1 and 1.5 or less
D: (Particle diameter of activated particles after ultrasonic dispersion)/(particle diameter of particles before activation)=more than 1.5 and 2.0 or less
E: A remaining sediment was visually observed.

The evaluation results of the activated particles of the particles 1 to 10 and the comparative particles 1 to 4 are shown in Table 2.

TABLE 2

| | Evaluation of redispersibility |
|---|---|
| Example 1 | A |
| Example 2 | A |
| Example 3 | A |
| Example 4 | B |
| Example 5 | B |
| Example 6 | B |
| Example 7 | B |
| Example 8 | C |
| Example 9 | C |
| Example 10 | B |
| Comparative Example 1 | D |
| Comparative Example 2 | D |
| Comparative Example 3 | D |
| Comparative Example 4 | E |

### [Evaluation 2] Evaluation of Nonspecific Adsorptivity with Respect to Particle

### (Evaluation of Chyle)

The particles 1 were dispersed in a phosphate buffer at 0.1 mass % to prepare a dispersion. Next, 60 μL of a chyle liquid formed of triolein, lecithin, free fatty acids, bovine albumin, and a Tris buffer was added to 30 μL of the dispersion, and the absorbance of the dispersion immediately after its stirring at a wavelength of 572 nm was measured. A spectrophotometer Biospectrophotometer manufactured by Eppendorf SE was used in the absorbance measurement. Then, the dispersion was left at rest at 37° C. for 5 minutes, and then its absorbance at a wavelength of 572 nm was measured again, followed by the calculation of a value of “(variation ΔABS in absorbance)×10,000.” The evaluation was performed based on the value of ΔABS×10,000 as described below.
AA: ΔABS×10,000 of 30 or less
A: ΔABS×10,000 of more than 30 and 50 or less
B: ΔABS×10,000 of more than 50 and 100 or less
C: ΔABS×10,000 of more than 100 and 500 or less
D: ΔABS×10,000 of more than 500

The evaluation result was AA. It was recognized that the particle according to the present invention was excellent in ability to suppress nonspecific adsorption.

### [Evaluation 3] Production of Test Particle by Antibody Sensitization of Particle and Evaluation of Agglutination Sensitivity of Test Particle

### (Production of Test Particle by Antibody Sensitization of Particle)

180 μL of a 1.7 mass % water suspension of the particles 1 was dispensed in a 1.5 mL microtube. 90 μL of a 5.0% aqueous solution of 1-(3-dimethylaminopropyl)-3-ethylcarbodiimide hydrochloride and 90 μL of a 5.0% aqueous solution of sodium N-hydroxysulfosuccinimide were added thereto. The mixture was stirred at room temperature for 30 minutes to provide a dispersion of activated particles (activated particle dispersion).

After centrifugal washing, 270 μL of phosphate buffered saline (hereinafter referred to as “PBS”) having a pH of 7.2 was added to the resultant, and the activated particles were dispersed with an ultrasonic wave.

5 μL of a 4.9 mg/mL dispersion of clone C5 (Funakoshi Co., Ltd.) of a monoclonal mouse anti-human C-reactive protein (hereinafter referred to as “CRP antibody”) was added thereto. The mixture was stirred at room temperature for 3 hours to provide test particles by antibody sensitization of the particles. Those test particles were subjected to centrifugal washing. After that, 1 mL of the PBS was added to the resultant, and the resultant was stored in a state of being dispersed.

### (Evaluation of Agglutination Sensitivity of Test Particle)

Standard serum for CRP was diluted with the PBS to a concentration of 0.75 mg/dL, and the resultant was used as a CRP specimen solution. A mixed liquid obtained by mixing 1 μL of the above-mentioned CRP specimen solution and 50 μL of a buffer (PBS containing 0.01% Tween 20) (hereinafter referred to as “mixed liquid 1”) was prepared and held at 37° C.

Next, 50 μL of the above-mentioned dispersion of test particles in which the test particles were sufficiently dispersed again with an ultrasonic wave before use (particle concentration: 0.1 mass %) was mixed with the mixed liquid 1. The absorbance at a wavelength of 572 nm of the mixed liquid (volume: 101 μL) immediately after stirring was measured. A spectrophotometer Biospectrophotometer manufactured by Eppendorf SE was used in the absorbance measurement. Then, the mixed liquid was left at rest at 37° C. for 5 minutes, and then its absorbance at a wavelength of 572 nm was measured again, followed by the calculation of the value of “(variation ΔABS in absorbance)×10,000.”

The value was calculated to be 10,000 or more, which was satisfactory as an evaluation result. It was recognized that the particle according to the present invention had a sufficient ability as a test particle.

According to the present invention, there are provided a particle and a test particle improved in redispersibility of the particle, in particular, in redispersibility thereof in the reactive functional group activation step in the case of chemically bonding a ligand to the particle surface.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2023-203386, filed Nov. 30, 2023, which is hereby incorporated by reference herein in its entirety.

What is claimed is:

1. A particle comprising a copolymer having a structural unit represented by the following formula (1) and a structural unit represented by the following formula (B),
wherein, when a structure obtained by removing R²² from the structural unit represented by the formula (B) is represented by B-X, a content of the B-X in the particle is 5.0 mass % or more and 17.5 mass % or less: 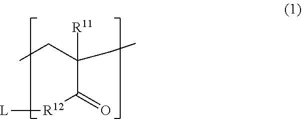
in the formula (1), L represents an unsubstituted or substituted divalent or more and hexavalent or less hydrocarbon group having 1 or more and 15 or less carbon atoms, which may have an oxy group, R¹¹ represents a hydrogen atom or a methyl group, and R¹² represents an oxygen atom or an imino group, and R¹¹, R¹², and L may vary depending on each structural unit; 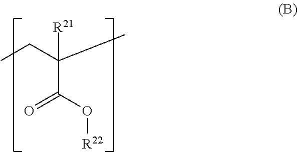
in the formula (B), R²¹ represents a hydrogen atom or a methyl group, R²² represents a group having an epoxy group, a group having a hydroxy group, or a group having a carboxy group or a salt thereof, and R²¹ and R²² may vary depending on each structural unit.

2. The particle according to claim 1, further comprising a structural unit represented by the following formula (A): 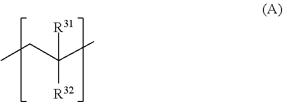
where R³¹ represents a hydrogen atom or a methyl group, R³² represents a substituted or unsubstituted phenyl group or a substituted or unsubstituted naphthyl group, a substituent thereof is a methyl group, an ethyl group, a hydroxy group, a carboxy group or a salt thereof, or a sulfo group or a salt thereof, and R³¹ and R³² may vary depending on each structural unit.

3. The particle according to claim 2, wherein, when a content of the structural unit represented by the formula (A) in the particle is represented by M(A), and a content of the B-X in the particle is represented by M(B), a value of a mass ratio M(B)/M(A) between the contents is 0.05 or more and 1.00 or less.

4. The particle according to claim 1, wherein the copolymer has the structural unit represented by the formula (1) in which the L represents any one of a structure represented by the following formula (L-1), a structure represented by the following formula (L-2), a structure represented by the following formula (L-3), a structure represented by the following formula (L-4), a structure represented by the following formula (L-5), a structure represented by the following formula (L-6), a structure represented by the following formula (L-7), a structure represented by the following formula (L-8), a structure represented by the following formula (L-9), a structure represented by the following formula (L-10), a structure represented by the following formula (L-11), a structure represented by the following formula (L-12), a structure represented by the following formula (L-13), a structure represented by the following formula (L-14), and a structure represented by the following formula (L-15): 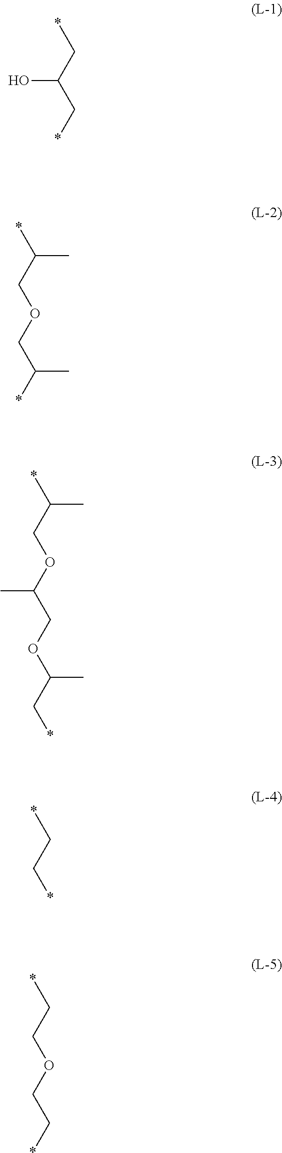 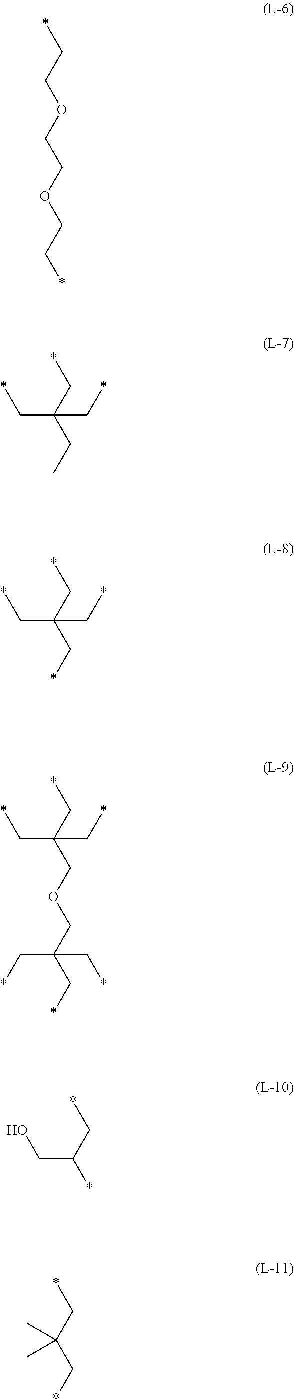 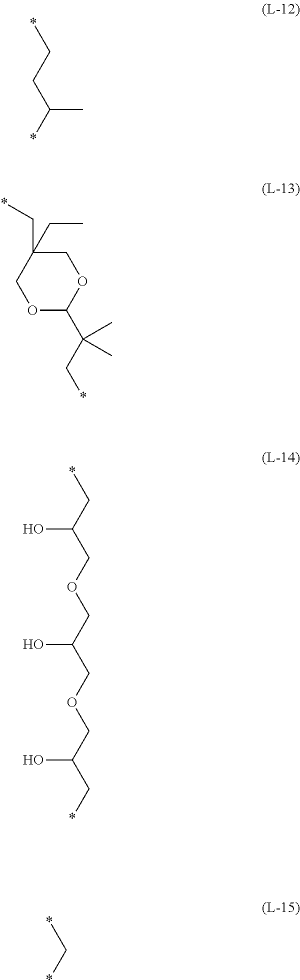
where “*” represents a bonding position with R¹² in the formula (1).

5. The particle according to claim 1, wherein the copolymer has at least any one of a structural unit represented by the following formula (1-1) and a structural unit represented by the following formula (1-2) as the structural unit represented by the formula (1),

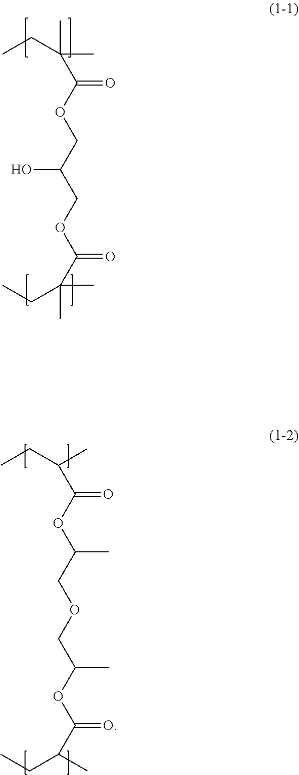

6. The particle according to claim 1, wherein a content of the structural unit represented by the formula (1) in the particle is 1 mass % or more and 10 mass % or less.

7. The particle according to claim 1, further comprising a compound having one of an amide structure, an amidine group, or an imidazoline structure.

8. A particle obtained by a production method including a polymerization step of performing a polymerization reaction of a reaction system containing at least any one of glycidyl methacrylate and glycidyl acrylate, a monomer represented by the following formula (G1), and a monomer represented by the following formula (A1),
wherein, in the polymerization step, a total content of the glycidyl methacrylate and the glycidyl acrylate with respect to a content of the monomer represented by the following formula (A1) is 10 mass % or more and 70 mass % or less: 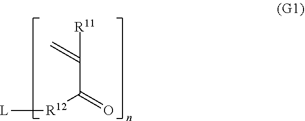
in the formula (G1), L represents an unsubstituted or substituted hydrocarbon group having 1 or more and 15 or less carbon atoms, which may have an oxy group, “n” represents an integer of 2 or more and 6 or less, R¹¹ represents a hydrogen atom or a methyl group, and R¹² represents an oxygen atom or an imino group; 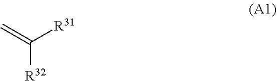
where R³¹ represents a hydrogen atom or a methyl group, R³² represents a substituted or unsubstituted phenyl group, or a substituted or unsubstituted naphthyl group, and a substituent thereof is a methyl group, an ethyl group, a hydroxy group, a carboxy group or a salt thereof, or a sulfo group or a salt thereof.

9. A particle obtained by a production method including a polymerization step of performing a polymerization reaction of a reaction system containing at least any one of glycidyl methacrylate and glycidyl acrylate and a monomer represented by the following formula (G1),
wherein, when a structure obtained by removing R²² from a structural unit represented by the following formula (B) derived from at least any one of the glycidyl methacrylate and the glycidyl acrylate in the particle is represented by B-X, a content of the B-X in the particle is 5.0 mass % or more and 17.5 mass % or less: 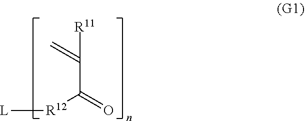
in the formula (G1), L represents an unsubstituted or substituted hydrocarbon group having 1 or more and 15 or less carbon atoms, which may have an oxy group, “n” represents an integer of 2 or more and 6 or less, R¹¹ represents a hydrogen atom or a methyl group, and R¹² represents an oxygen atom or an imino group; 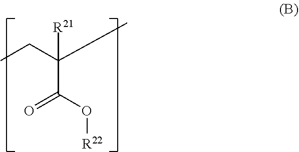
in the formula (B), R²¹ represents a hydrogen atom or a methyl group, R²² represents a group having an epoxy group, a group having a hydroxy group, or a group having a carboxy group or a salt thereof, and R²¹ and R²² may vary depending on each structural unit.

10. The particle according to claim 8, wherein the polymerization step is performed by using a water-soluble polymerization initiator.

11. The particle according to claim 10, wherein the water-soluble polymerization initiator has one of an amide structure, an amidine group, and an imidazoline structure.

12. The particle according to claim 9, wherein the polymerization step is performed by using a water-soluble polymerization initiator.

13. The particle according to claim 12, wherein the water-soluble polymerization initiator has one of an amide structure, an amidine group, and an imidazoline structure.

14. A test particle comprising a ligand added to a surface of the particle of claim 1.

15. A reagent comprising the test particle of claim 14 dispersed in an aqueous solution.

16. A test kit comprising the reagent of claim 15 and a case configured to enclose the reagent.

17. A method of detecting a target substance in a specimen, the method comprising mixing the reagent of claim 15 and the specimen that may contain the target substance.

18. A method of detecting a target substance in a specimen by an agglutination method, the method comprising:
mixing the reagent of claim 15 with a specimen that may contain the target substance to provide a mixed liquid;
irradiating the mixed liquid with light; and
detecting at least any one of transmitted light and scattered light from the light with which the mixed liquid is irradiated.
